(19) United States
(12) Patent Application Publication
(10) Pub. No.: US 2022/0265200 A1
(43) Pub. Date: Aug. 25, 2022

(54) COMPUTER IMPLEMENTED METHOD TO ALLOW THE IMMUNE SYSTEM OF A PATIENT TO BETTER FUNCTION

(71) Applicant: Gaia AG, Hamburg (DE)

(72) Inventor: Mario Weiss, Hamburg (DE)

(21) Appl. No.: 17/181,503

(22) Filed: Feb. 22, 2021

(51) Int. Cl. A61B 5/00; G16H 50/20

(52) U.S. Cl. CPC A61B 5/414; A61B 5/7475; A61B 5/4839; G16H 50/20

(57) ABSTRACT

The present invention relates to a computer implemented method for allowing the immune system of a patient to better function, said method comprising: gaining patient input data from the patient by presenting at least one page through an interface to the patient together with selection possibilities, assessing a patient specific psychological and/or social and/or medical status including the communication type of the patient based on at least one selection of the patient to the page, assembling of a patient specific content and presenting it in a patient status sensitive, tailored way in a communication flow wherein the communication flow includes a set of pages and selection possibilities of the patient.

### TECHNICAL FIELD

The present invention relates to a computer implemented method to allow the immune system of a patient to a better function as well to a method for treating a patient with an auto immune disease, cancer or an infection disease. In addition, the invention can be used to prevent such a disease. In addition, the present invention is directed at a device for conducting a respective method.

### SUMMARY

The method is a computer implemented method and allows the immune system of a patient to better function.

The method includes gaining patient input data by presenting at least one page through an interface to the patient together with selection possibilities. The at least one page is presented by means of the interface, preferably a monitor, further preferred together with speakers and/or a microphone in communication with the interface, wherein the monitor is preferably the screen of an electronic device preferably a mobile electronic device such as for example a laptop, a mobile phone or a wearable electronic device, such as a watch. In particular, the computer implemented method is configured as an application for an electronic device. The at least one page serves for an assessment of the patient based on its selections.

The method further comprises assessing a patient specific psychological and/or social and/or medical status including the communication type of the patient based on at least one selection of the patient to the at least one page. This patient specific content is presented it in a patient status sensitive, tailored way in a communication flow by means of a set of pages and selection possibilities of the patient. The content presented in the communication flow and aims at triggering an effect, in particular balancing effect, on the immune system. The patient specific content is presented as described above regarding the at least one page.

Preferably the method is used for treating a patient suffering from an autoimmune disease, cancer or an infection disease by improving its immune system to better function. It can also be used to prevent diseases to occur or to occur with lower intensity if they occur.

The at least one page presented to the patient preferably includes an image and/or audio file and/or text block. In particular, the method includes creating the page out of a plurality of images, audio files and/or text blocks.

The page is accompanied with selection possibilities of the patient. The selection of the patient based on the at least page serves to capture the psychological and/or social and/or medical status of the patient including the communication type of a patient. The selection can reflect on the preferences of the patient on how the patient would like to be addressed, on a logical or in other words cognitive or emotional level. For example, shy or introvert patients can prefer to be addressed differently then extrovert patients. It can also reflect on how a behavioral therapist would address such a patient to trigger behavioral change or to change mental thought patterns.

The at least one page can be aimed to find out opinions and/or experiences of the patient. For example, the at least one page can include questions relating to whether the patient knows and/or has experienced an interaction between cognition, emotions and actions. If selections of the patient indicate that the patient is not aware of such an interaction, further pages can include suggestions for the patient to mesmerize situation in which the patient has for example experienced an interaction, e.g. an amended behavior influenced by emotions, in order to create a respective awareness by the patient. If the patient on the other hand is well aware of the interaction, respective pages to make the patient understand such a interaction do not need to be created and presented to the patient.

Assessing the patient's status and communication type, which can be different between patients and can also change regarding the same patient over time and situation, serves to present the patient with an optimal communication content in the optimal sequence to which the patient reacts best in a therapeutic desired way at the point of time of system usage. This a crucially important since otherwise the patient does not respond as well to the presented method.

Furthermore, the at least one page can include questions to gain more patient input data such as a disease from which the patient suffers and/or diagnosis. Furthermore, questions can relate to the severity of the disease and/or diagnosis and/or symptoms. The questions can also relate to a mental state and/or a physical state of the patient. Furthermore, the questions can concern effects or side effects of a drug which the patient takes. This additional patient input data which can be referred to as medical patient input data, can serve to assess the overall state of the patient.

Medical patient input data can also be included in the assembly of the patient content which is to be presented in a communication flow. The patient content as well as the communication flow might furthermore be dependent on the disease and/or diagnosis and/or on the expectations and/or goals of the patient using the computer implemented method.

The term communication flow is to be understood as the sequence in which the patient content is presented to the patient in the form of pages including e.g. at least one text block and/or audio file and/or image as explained together with respective selection possibilities. After each selection a new page is presented to the patient wherein the method includes determining the next page based on the previous selection as well as rules which are generated and/or selected for this individual patient. The rules are a set of if-then rules which can be sequenced, activated and/or deactivated by other if-then rules.

These rules can be predefined. For example, the rules can define a pre-defined next page for each possible selection. Further, the rules can also be dynamically created by the system via machine learning algorithms like neuronal networks, regression models or Bayesian filtering/logic. The pages presented in the communication flow are thus connected by pre-defined or dynamically created if-then-rules. The communication flow might also depend on a specific therapy concept.

At first, the at least page presented to the patient before the assessment can only be aimed at gaining patient input data to be able to evaluate the patient, thus its psychological and/or social and/or medical status including the communication type of the patient. For this purpose, the patient can be grouped to a specific predefined patient content and predefined communication flow defined by a rough hypothesis of the optimal fit by the patient input data. In other words, a patient content and communication flow can be chosen from a predefined selection by means of the interaction of the patient to the at least one page.

Then the patient content is presented to the patient by means of the communication flow. The pages of the patient content preferably include an image and/or audio file and/or text block. In particular, the method includes creating each page out of a plurality of images, audio files and/or text blocks. The assembly of the content is trigged by if-then rules and the content depends on the communication flow and the selections which have already been made by the patient. Pages that are presented to the patient can include, for example by means of a text block, instructions or suggestions to the patient which can for example be aimed at achieving a behavioral change, for example a motivation for a physical exercise and/or a change of diet and/or a change in sleeping behavior. As explained before, the patient content can be dependent on a specific situation of the patient, e.g. its mental situation, which can be assessed by the patient input data or by following selections to pages. The selection which image and/or audio file and/or text block and/or motivation can depend on predefined rules or cases. The patient content presented in the communication flow aims at triggering an effect on the immune system to better function and thus treating or preventing the above named diseases.

Alternatively, or additionally the communication flow, thus the sequence of presenting the patient content, cannot only be rule-based as explained above but it can also be case-based. For example, based on the gained patient input data the patient can be assigned to a predefined case which is most similar and benefited highly from already existing case data. In detail, the computer implemented method can include a plurality of predefined cases which include patient content and communication flows which best fit to certain patient input data and/or sensor input data. These cases can for example be evaluated and labeled by a professional, such as a doctor or psychotherapists, as successfully treated wherein the labels can include an assessment of the development of the patient, e.g., an improvement of symptoms of the underlying disease.

The effect on the immune system is preferably a balancing one, especially in order to treat an autoimmune disease, cancer or an infection disease as explained above. With balancing it is referred to the optimal status of an immune system that is highly reactive and sensitive against for example cancer, bacteria or viruses but at the same time does not overreact to destroy healthy cells of the body. The objective is to reduce the occurrence of body states that are for example described as “chronic inflammation”, thus a situation where the immune system has overacted for a longer period and has lost its sensitivity and flexibility to react adaptively and specifically to external pathogens like bacteria or viruses or cancer cells. In addition a better balanced immune system is of high value in so called autoimmune diseases like rheumatoid arthritis, multiple sclerosis, psoriasis or colitis ulcerosa/morbus chron. A better balanced immune system is furthermore of help to prevent diseases, e.g. increase the effect of vaccines on teaching the immune system to build an immunological defense against viruses, bacteria or cancer cells.

The patient content, thus the set of pages which is shown to the patient in the communication flow, serves to stimulate the vagus nerve which can either be achieved directly by the page itself or indirectly by a suggested behavioral change. In addition or alternative the so called HPA (Hypothalamic-pituitary-adrenal axis), the vegetative system or the cytokine system can be triggered.

The vagus nerve is connected to the parasympathetic system and influences its activity along with other body functions such as for example the heart, the blood pressure and the respiration. The vagus nerve influences the immune response especially the inflammatory response, in other words the cytokine and T cell production. Inflammation is in many cases the body's response to a stressor.

The stimulation of the vagus nerve can inhibit overreacting cytokine productions and this balances the immune systems normalizing its response to new triggers as well as reducing the symptoms related to a hyperactive immune system.

The balancing effect can be achieved by the patient content in the scope of the communication flow, e.g. since the shown pages can have a stress reducing, calming effect on the patient. As an example, a page can contain an image of a wide landscape which has a calming effect and/or an audio file with stress reducing sounds such as e.g. the murmur of waves. The pages can also show the patient for example a tailored exercise that can activate a vagal reaction or they can convince the patient to change behavior for example to conduct physical activities result in stimulating the lymph which then results in a better distribution of lymphatic cells improving the immune response. The method thus exposes the patient to specific tailored content, e.g. in the form of an image and/or audio file and/or a text block. The method can also trigger behavioral change that actives immune reaction for example via vagal or HPA axis. Such change could be performing exercises or using specific though modeling methods to reduce stress. In this way, the patient is treated or a development of the above named diseases is prevented.

The method can include gaining sensor input data wherein the sensor input data is considered in assembling the patient content and presenting it in the communication flow. The sensor input data can preferably include medical characteristics, such as for example the heart rate or the heart rate variability. The latter is especially important since it is directly coupled to the immune system by the parasympathetic system. For example, the higher the heart rate variability is, the more active the parasympathetic system that has a balancing effect on the immune response. Another medical characteristic can for example be the weight or the blood pressure of the patient but also laboratory data like immune cell counts or status.

The consideration of the sensor input data in assembling the patient content and presenting it in the communication flow can be established by the fact that sensor input data is an input for at least one of the rules. As an example, a rule can be dependent on the value of the heart rate variability. If it is above or below a pre-defined threshold for a pre-defined time, a rule can be defined to amend the communication flow or the patient content.

The method can further include gaining further patient input data by assessing the selections of the patient to the presented patient content. This especially serves to monitor the development of the patient and the success of the method or in other words the treatment. Furthermore, additional sensor input data can be gained.

By reacting to the content, the method is gathering more information about the patient. This information is used to create new rules and/or to active and/or deactivate existing rules. These rules are then used to determine what kind of information, namely content of the pages of the patient content, in what kind of sequence will be presented to the patient. So, the patient content and the communication flow can be adapted. The adaptation of the patient content is preferably dynamic and further preferred in real-time. On the adapted patient content and communication flow further patient input data can be gained based on a new communication flow to then reassess the former. Thus, the development and the best fit to the patient's status, development and the overall treatment is monitored regularly. The assembly of the patient content and the communication flow is thus dynamic and is adapted in real time. Also, by means of further gained patient input data and sensor input data or other medical data, the development of the patient, and thus the treatment, is monitored regularly.

The additional data can be gained at different points in time such as for example after a certain time period has passed or a certain number of pages has been presented.

In other words, based on the additional input data it can be assessed whether the patient generally responds well to patient content and the communication flow. Furthermore, it can be assessed whether the desired improving effect on the immune system is achieved, e.g. by monitoring the heart variability rate, cell activity or other inflammation parameters.

In another aspect the method aims at treating a patient with an autoimmune disease, an infection disease or cancer, wherein the method comprises the step of administering to the patient a drug, such as e.g. a pharmaceutical or biological drug, or a vaccine. The drug or the vaccine are aimed at treating or preventing the disease.

In an autoimmune disease scenario as well as conducting an above described computer implemented method. The drug can include antibodies and/or interleukins, such as for example TNF inhibitors or JAK inhibitors. However, these drugs only have a specific effect on a particular point in the complicated cascade of the immune system and are not successful with regard to completely treating or substantially reducing the symptoms of the above-described diseases, since only about 50% to 70% of the patients respond well enough. In addition a lot patients will suffer from drug side effects as the dosage of the drug has to be high to show effects. In addition a lot of patients will stop using the drug after a couple of years even if lifelong usage is indicated. By providing the patient with a bundle comprising the drug as well as the computer implemented method this gap in reaction to the drug can be closed as well as the effect of the drug can be increased. This can then lead to less drug needed with less drug associated side effects. This can then lead to longer drug tolerance and usage. This is due to the effect that the computer implemented method balances the immune system as described above and thus improves the overall effect of the drug since its effect can be much higher if it encounters a balanced immune system.

In cancer modern drugs try to influence the metabolism of cancer cells to block the proliferation of these cells. After the proliferation of these cells is reduced, the immune system of the patient has to destroy the remaining pathological cells. Experimental immunotherapy cancer treatments are trying to train the immune system better to kill cancer cell more effectively. Also, in this context a balanced and therefore better working immune system plays an important role to support the drug effect. The same mechanism applies for infectious diseases like bacterial infections or virus infections and also vaccination processes.

The method can comprise the step of assessing the dosage of a pharmaceutical drug or biological drug based on the gained patient info data or sensor input data and possibly suggesting an amendment of the dosage based on the assessment of the patient and/or a responsible doctor. The patient can be for example be given a suggestion, for example based on sensor input data such as the weight, to seek the advice of a doctor in order to discuss an assessment of the dosage of the drug. Furthermore, the method can comprise sending a respective alert to the patient and/or the respective doctor. Beside the dosage the method can also give advice to change the drug administered to another drug with higher effectiveness or lower side effects for the individual patient profile that has been revealed by the data gathered in the interaction with the patient. This helps to understand so called “non responding patients”.

In another aspect the invention is directed towards a device for conducting an above-described method when the device includes an assessment unit for assessing the patient content and for presenting the content in a patient status sensitive, tailored way in a communication flow. The device, especially the assessment unit, can comprise a neural network which can determine the patient content as well as the communication flow. It thus determines the content as well as the exact sequence of pages to be displayed to the patient which can be rule-based or case-based. Further, the neural network evaluates the selections of the patient. In particular, the neural network is assembly the patient content and the communication flow based on predefined if-then-rules and/or predefined cases. The assessment unit can be configured to follow instructions to identify the next page or the respective components of the page to collect more input data and/or to trigger a positive effect on the immune system.

The device can further include a storage medium storing images and/or text blocks and/or audio files to prepare pages to be presented to the patient. The storage medium can further include predefined if-then-rules and/or predefined cases. The storage medium can store the selection of the patient to the pages, preferably including at least an image and/or audio file and/or text block.

The device further includes a processor for carrying out the above described computer implemented method. The device can further include at least one sensor to collect sensor input data.

Furthermore, the invention relates to computer program product, comprising a computer-readable storage medium on which a program is stored, which, after being loaded into the memory of a computer, enables the computer to carry out an above described method in cooperation with an above described device.

In addition, the invention relates to a computer-readable storage medium on which a program is stored, which, after being loaded into the memory of the computer, enables the computer to carry out an above described method in cooperation with an above described device.

### BRIEF DESCRIPTION OF THE DRAWINGS

The above-mentioned and the other features of the invention disclosed herein are described below with reference to the drawings of the preferred embodiments. The illustrated embodiments are intended to illustrate, but not to limit the inventions. The drawings contain the following figures:

FIG. 1: a schematic flow chart of a method according to the invention; and

FIG. 2: a schematic overview of a device according to the invention.

### DETAILED DESCRIPTION

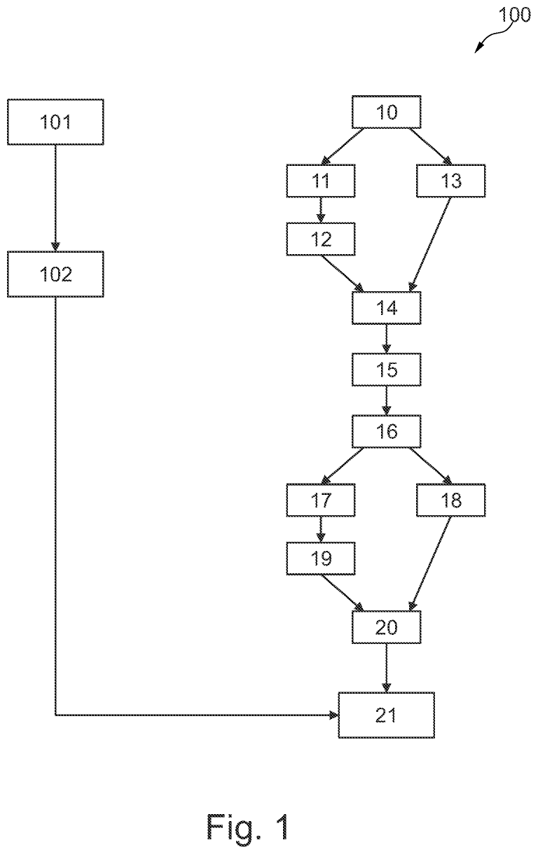

FIG. 1 shows a schematic flow chart of a computer implemented method 10 according to the invention as well as of a method 100 for treating a patient with an autoimmune disease, an infection disease or cancer. Both methods can be used to prevent such a disease.

The method 100 can include administering 101 to the patient a drug or a vaccine aimed at treating or preventing an above named disease.

In parallel, the method 100 can include conducting the computer implemented method 10 which will be described in the following. The computer implemented method 10 includes gaining 11 patient input data by presenting 12 at least one page through an interface to the patient together with selection possibilities. Further, the method 10 can include gaining 13 sensor input data by means of a sensor. The method 100 can further include assessing 14 a communication type as well as a patient specific psychological and/or social and/or medical current status in addition to a preferred outcome (by the patient and/or the therapy concept). This input is based on the at least one selection to the at least one page. Based on the assessment as well as preferably on the sensor input data a patient content will be assembled 15 which is aimed a triggering an effect, especially a balancing effect, on the immune system. The balancing effect aims at bringing an imbalance, such as e.g. a hyperactive state of a conically inflamed immune system, back into its normal response range. Once a patient content is assembled it is presented 16 by pages according to a communication flow. Further patient input data is gained 17 and the respective selections of the patient can be assessed 19. In addition, further sensor input data can be gained 18. Based on the newly gained input data the patient content can dynamically be adapted 40.

The method 100 for treating a patient with an autoimmune disease, an infection or cancer can include assessing 102 the dosage of a drug based in the gained patient input data or sensor input data.

Overall, either by conducting the computer implemented method alone or as part of a method 100 for treating a patient with an autoimmune disease, cancer or an infection disease the patient is treated or such a disease is prevented 41.

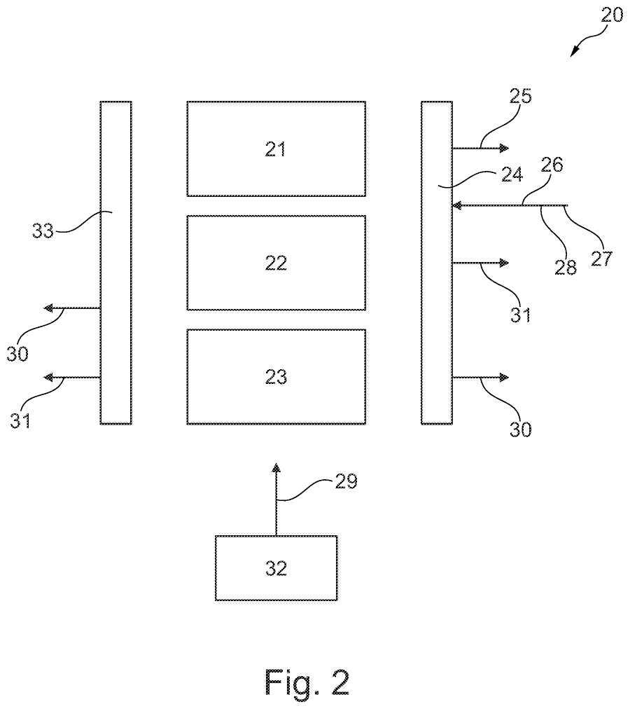

FIG. 2 shows the schematic overview of a device according to the present invention. The device 20 comprises an assessment unit 21, a storage medium 22 and a processor 23. The processor 23 allows the assessment unit 21 to function.

The device 20 further comprises an interface 24 with a patient and can further comprise an interface 33 with the doctor. By means of the interface 24 to the patient pages 25 can be presented. Based on these pages 25 patient input data 26 can be gained. In addition, the device 20 can comprise a sensor 23 to collect sensor input data 29. By means of the interfaces the device 20 can send suggestions 31 or alerts 30 to the patient or the doctor.

While the present disclosure has been illustrated and described with respect to a particular embodiment thereof, it should be appreciated by those of ordinary skill in the art that various modifications to this disclosure may be made without departing from the spirit and scope of the present disclosure.

What is claimed is:

1. A computer implemented method for allowing the immune system of a patient to better function, said method comprising:
gaining patient input data from the patient by presenting at least one page through an interface to the patient together with selection possibilities,
assessing a patient specific psychological and/or social and/or medical status including the communication type of the patient based on at least one selection of the patient to the page, and
assembling a patient specific content and presenting it in a patient status sensitive, tailored way in a communication flow,
wherein the communication flow includes a set of pages and selection possibilities of the patient.

2. The method according to claim 1, wherein the method is used for treating a patient suffering from an auto immune disease, an infection disease or cancer or preventing such a disease.

3. The method according to claim 1,
wherein the method includes gaining sensor input data, and
wherein the sensor input data is considered in assembling the patient specific content and presenting it in a patient status sensitive, tailored way in the communication flow.

4. The method according to claim 1,
wherein the method includes gaining further patient input data by assessing
selections of the patient to the patient specific content.

5. The method according to claim 3, wherein the method also includes gaining further sensor input data.

6. The method according to claim 4,
wherein the method includes a dynamic adaption of the patient specific content and/or the communication flow based on the further patient input data and/or the further sensor input data.

7. A method for treating a patient with an auto immune disease, an infection disease or cancer or for preventing the patient from developing such a disease, said method comprising the steps of:
administering to the patient a drug or a vaccine, said drug or vaccine aimed at treating or preventing the disease or cancer,
wherein the method further comprises conducting a computer-implemented method according to claim 1.

8. The method according to claim 7, wherein the method comprises the steps of:
assessing the dosage of the drug based on gained patient input data or sensor input data and possibly suggesting an amendment of the dosage based on the assessment to the patient and/or a doctor.

9. A device for conducting a method according to claim 1, wherein the device includes an assessment unit, said assessment unit configured to assemble a patient specific content and to present the content in a patient status sensitive, tailored way in a communication flow.

10. (canceled)

11. A non-transitory computer-readable storage medium on which a program is stored, which, after being loaded into the memory of a computer, enables the computer to carry out a method for allowing the immune system of a patient to better function, said method comprising:
gaining patient input data from the patient by presenting at least one page through an interface to the patient together with selection possibilities,
assessing a patient specific psychological and/or social and/or medical status including the communication type of the patient based on at least one selection of the patient to the page, and
assembling a patient specific content and presenting it in a patient status sensitive, tailored way in a communication flow,
wherein the communication flow includes a set of pages and selection possibilities of the patient
in cooperation with a device having an assessment unit, said assessment unit configured to assemble a patient specific content and to present the content in a patient status sensitive, tailored way in a communication flow.

12. The method according to claim 5, wherein the method includes a dynamic adaption of the patient specific content and/or the communication flow based on the further sensor input data.
